(19) United States
(12) Patent Application Publication
(10) Pub. No.: US 2019/0000486 A1
(43) Pub. Date: Jan. 3, 2019

(54) EXPANDABLE OCCLUSION DEVICES, SYSTEMS, AND METHODS

(71) Applicant: COVIDIEN LP, Mansfield, MA (US)

(72) Inventor: SCOTT J. PRIOR, SHELTON, CT (US)

(21) Appl. No.: 15/942,791

(22) Filed: Apr. 2, 2018

Related U.S. Application Data
(60) Provisional application No. 62/526,454, filed on Jun. 29, 2017.

(51) Int. Cl. A61B 17/12; A61B 17/42; A61B 17/34; A61B 17/02; A61L 31/14

(52) U.S. Cl. CPC A61B 17/12181; A61B 17/4241; A61B 17/3423; A61B 17/0218; A61B 2017/00871; A61B 17/12099; A61L 31/14; A61B 2017/345; A61B 2017/3484; A61B 17/1204

(57) ABSTRACT

An expandable occlusion device is provided including an expandable occluder expandable from a compressed state to an expanded state to sealingly engage tissue. An outer sheath that defines a longitudinal axis may be disposed about the expandable occluder. The outer sheath may be configured to compress the expandable occluder and maintain the expandable occluder in the compressed state. A pull cord may be operably coupled to the outer sheath and selectively manipulatable to tear the outer sheath such that the outer sheath is removed from the expandable occluder allowing the expandable occluder to expand from the compressed state to the expanded state.

### CROSS REFERENCE TO RELATED APPLICATION

The present application claims the benefit of and priority to U.S. Provisional Application Ser. No. 62/526,454, filed on Jun. 29, 2017 the entire contents of which are incorporated herein by reference.

### TECHNICAL FIELD

The present disclosure relates to surgical devices, systems, and methods and, more particularly, to expandable occlusion devices, systems, and methods for maintaining pneumoperitoneum or another seal during a surgical procedure.

### BACKGROUND

Sealing a surgical opening, e.g., a natural orifice or surgically-created opening, is often necessary in order to maintain pneumoperitoneum or for other purposes. During a laparoscopic hysterectomy, for example, the abdominal cavity is insufflated with a gas, or pneumoperitoneum, to distend and separate the abdominal wall from the uterus, such that the clinician can adequately view the surgical site and perform the hysterectomy. Laparoscopic hysterectomy is typically performed using a uterine manipulator including an occluder balloon that can be inflated within the vaginal cavity to prevent air from escaping and creating a loss of pneumoperitoneum. During the course of a laparoscopic hysterectomy, it may be necessary to introduce and withdraw the uterine manipulator multiple times, at least to create a seal to maintain pneumoperitoneum at various different points during the laparoscopic hysterectomy. Reintroducing the uterine manipulator into the vaginal cavity and re-inflating the occluder balloon multiple-times may be unnecessarily invasive.

### SUMMARY

Accordingly, a need exists for occluding devices, systems, and/or methods for maintaining pneumoperitoneum or other seals during surgical procedures.

According to an aspect of the present disclosure, an expandable occlusion device is provided, including an expandable occluder expandable from a compressed state to an expanded state to sealingly engage tissue. An outer sheath may define a longitudinal axis and may be disposed about the expandable occluder. The outer sheath may be configured to compress the expandable occluder and maintain the expandable occluder in the compressed state. A pull cord may be operably coupled to the outer sheath and selectively manipulatable to tear the outer sheath such that the outer sheath is removed from the expandable occluder allowing the expandable occluder to expand from the compressed state to the expanded state.

In embodiments, an application of tensile force to the pull cord tears the outer sheath and removes the outer sheath from the expandable occluder.

In some embodiments, the expandable occlusion device may include a release feature associated with a surface of the outer sheath and coupled to the pull cord. When a tensile force is applied to the pull cord, a corresponding tensile force is applied to the release feature. The application of tensile force to the release feature tears the outer sheath to form an opening in the outer sheath to facilitate removal of the outer sheath from the expandable occluder.

In certain embodiments, the release feature is selected from the group consisting of tear tape, peel tape, and shrink tape.

In embodiments, the release feature includes a series of perforations defined through a surface of the outer sheath.

In some embodiments, the outer sheath is formed from a biocompatible film.

In certain embodiments, the expandable occluder is formed from a closed-cell memory foam.

In embodiments, the pull cord includes first and second end portions, the first end portion fixedly attached to the outer sheath, the second end portion having a handle attached thereto, the handle configured for gripping the pull cord.

In some embodiments, one of the outer sheath and the expandable occluder includes a lubricious coating, the lubricious coating configured to aid at least one of insertion into a body cavity, removal from a body cavity, or removal of the outer sheath from the expandable occluder.

In certain embodiments, the expandable occlusion device defines a circular cross-sectional shape.

In embodiments, the expandable occlusion device is configured to receive a surgical instrument therethrough.

In some embodiments, the surgical instrument is a uterine manipulator.

According to another aspect of the present disclosure, a uterine manipulator is provided, including a handle, a shaft extending distally from the handle, an end effector assembly disposed at a distal end portion of the shaft, and an expandable occlusion device supported on the shaft.

The expandable occlusion device may include an expandable occluder expandable from a compressed state to an expanded state to sealingly engage tissue. An outer sheath may define a longitudinal axis and may be disposed about the expandable occluder. The outer sheath may be configured to compress the expandable occluder and maintain the expandable occluder in the compressed state. A pull cord may be operably coupled to the outer sheath, wherein the pull cord is selectively manipulatable to tear the outer sheath such that the outer sheath is removed from the expandable occluder allowing the expandable occluder to expand from the compressed state to the expanded state.

In embodiments, the pull cord of the expandable occlusion device is of sufficient length to extend proximally outward from a vaginal cavity when the uterine manipulator is inserted into the vagina cavity.

In some embodiments, when a tensile force is applied to the pull cord when the uterine manipulator is inserted into the vaginal cavity, the outer sheath is removed proximally outward from the vaginal cavity and the expandable occluder expands outwardly against the vaginal cavity to the expanded state to form a seal therein.

In certain embodiments, a release feature is associated with a surface of the outer sheath and coupled to the pull cord. When a tensile force is applied to the pull cord, a corresponding tensile force is applied to the release feature. The application of tensile force to the release feature tears the outer sheath to form an opening in the outer sheath to facilitate removal of the outer sheath from the expandable occluder.

According to another aspect of the present disclosure, a method for performing a surgical procedure is provided, including positioning an expandable occlusion device within a surgical cavity in a compressed state, the expandable occlusion device including an expandable occluder, an outer sheath disposed about the expandable occluder to maintain the expandable occluder in the compressed state, and a pull cord operably coupled to the outer sheath. A tensile force may be applied to the pull cord of the expandable occlusion device to remove the outer sheath from the expandable occluder such that the expandable occluder expands from the compressed state to an expanded state within the vaginal cavity to form a seal therein. A surgical site may be insufflated with a gas to create a pneumoperitoneum therein.

In some embodiments, a uterus may be positioned with a uterine manipulating tip portion of the uterine manipulator for at least one of retroversion and anteversion of the uterus.

In certain embodiments, a circular incision may be made into the uterus to separate at least part of the uterus from a vagina. The uterine manipulator may be withdrawn proximally from the vaginal cavity with at least part of the uterus attached to the uterine manipulating tip portion of the uterine manipulator.

In embodiments, a second expandable occlusion device may be positioned within a surgical cavity in a compressed state, the second expandable occlusion device including an expandable occluder, an outer sheath disposed about the second expandable occluder to maintain the second expandable occluder in the compressed state, and a pull cord operably coupled to the outer sheath. A tensile force may be applied the pull cord of the second expandable occlusion device to remove the outer sheath from the second expandable occluder such that the second expandable occluder expands from the compressed state to an expanded state within the vaginal cavity to form a seal therein. A surgical site may be insufflated with a gas to create a pneumoperitoneum therein.

### BRIEF DESCRIPTION OF THE DRAWINGS

Objects and features of the present disclosure will become apparent to those of ordinary skill in the art when descriptions thereof are read with reference to the accompanying drawings, of which:

FIG. 1 is a side view of an expandable occlusion device in a compressed state in accordance with the present disclosure;

FIG. 2 is cross-sectional view of the expandable occlusion device of FIG. 1 taken along section line A-A of FIG. 1;

FIG. 3 is a schematic diagram of the expandable occlusion device of FIG. 1, disposed within a vaginal cavity in the compressed state;

FIG. 4 is a schematic diagram of the expandable occlusion device of FIG. 1 disposed within the vaginal cavity in an expanded state;

FIG. 5 is a side view of a uterine manipulator including an expandable occlusion device in accordance with the present disclosure;

FIG. 6 is a schematic diagram of a distal end portion of the uterine manipulator of FIG. 5 disposed within the vaginal cavity with the expandable occlusion device in a compressed state; and

FIG. 7 is a schematic diagram of the distal end portion of the uterine manipulator of FIG. 5 disposed within the vaginal cavity with the expandable occlusion device in an expanded state.

### DETAILED DESCRIPTION

Embodiments of the present disclosure are described in detail with reference to the drawings in which like reference numerals designate identical or corresponding elements in each of the several views. As used herein, the term “distal” refers to that portion of structure farther from the user, while the term “proximal” refers to that portion of structure closer to the user. As used herein, the term “clinician” refers to a doctor, nurse, or other care provider and may include support personnel. In the following description, well-known functions or constructions are not described in detail to avoid obscuring the present disclosure in unnecessary detail.

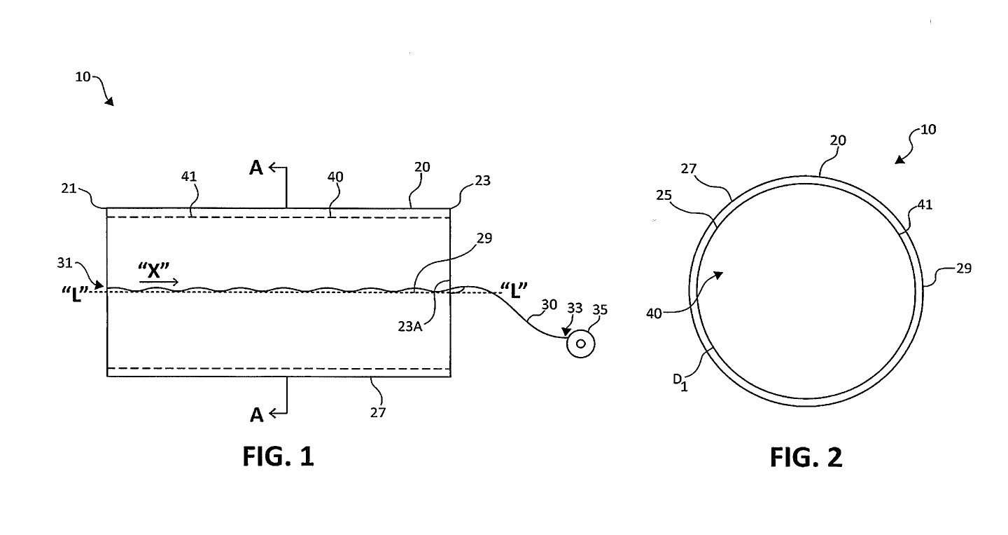

Referring to FIGS. 1 and 2, an expandable occlusion device is shown generally identified by reference numeral 10. Expandable occlusion device 10 defines a longitudinal axis “L” and generally includes an outer sheath 20, a pull cord 30 coupled to outer sheath 20, and an occluder 40 disposed within outer sheath 20.

Outer sheath 20 is generally configured to constrain occluder 40 in a first, compressed state to reduce the size (e.g., an outer dimension) of occluder 40. Outer sheath 20 includes respective first and second end portions 21, 23, an inner surface 25 in contact with occluder 40, and an outer surface 27 configured for slidable positioning within an artificial or natural opening of a patient (e.g., a body orifice).

Outer sheath 20 further includes a release feature 29 disposed along a length or surface thereof configured to aid in removing outer sheath 20 from occluder 40. Release feature 29 may include a plurality of perforations extending through respective inner and outer surfaces 25, 27 of outer sheath 20. Release feature 29 may alternatively or additionally include a peel tape, a shrink tape, a tear tape, or the like. When a sufficient tensile force is applied to release feature 29, release feature 29 tears outer sheath 20 along an at-least partially pre-determined path such that an opening (not explicitly shown) is formed in outer sheath 20 along longitudinal axis “L,” although paths that are angled relative to longitudinal axis “L,” helically wound about longitudinal axis “L,” etc., are also contemplated. Release feature 29 may be disposed along a partial length, or alternatively, a full length of outer sheath 20.

Outer sheath 20 may be any type of tube, sleeve, wrapping, film, or covering configured to constrain occluder 40 in a compressed state. For example, outer sheath 20 may be formed from a shrinkwrap, peelwrap, heatwrap, or similar material such as low-density polyethylene, polypropylene, polyesters, polyethylene terephthalate, polycarbonate, polyethylene, naphthalate, polyvinyl chloride, polystyrene, polytetrafluoroethylenepolyamide, fluorinated ethylene propylene, or the like. Outer sheath 20 may be formed from a flexible material and/or a material having a low thickness (e.g., 35-380 microns). Respective inner and outer surfaces 25, 27 of outer sheath 20 may be formed from, or coated with, a lubricious material or substance for easily removing outer sheath 20 from occluder 40 and/or for low friction insertion into a body orifice. Outer sheath 20 may be formed from paper or paperboard, paper laminates, metal or metalized film, foil, or the like. Although outer sheath 20 is shown as having a cylindrical shape, outer sheath 20 may be configured with any suitable shape, such as rectangular, square, triangular, elliptical, trapezoidal, or the like. In embodiments, outer sheath 20 may include more than one material and/or may be a combination of materials.

Pull cord 30 is fixedly attached to outer sheath 20 and/or release feature 29 and is configured to aid in removing outer sheath 20 from occluder 40. Specifically, when a sufficient tensile force is applied to pull cord 30, outer sheath 20 is torn, as detailed above, to create the opening (not explicitly shown) in outer sheath 20 such that outer sheath 20 can be removed from occluder 40. Pull cord 30 includes a first end portion 31 attached to outer sheath 20 and/or release feature 29 and a second end portion 33 attached to a handle 35 configured for gripping pull cord 30. Pull cord 30 may be formed from any suitable material configured to withstand a tensile force without tearing, such as, e.g., cotton, rayon, etc.

Pull cord 30 may be attached to any suitable portion or portions of outer sheath 20 such as, for example, inner and/or outer surfaces 25, 27 thereof. Pull cord 30 may be additionally or alternatively be operatively and/or fixedly connected, attached, integrated, or otherwise formed with release feature 29, as shown in FIG. 1. When a sufficient tensile force is applied to pull cord 30, pull cord 30 tugs at release feature 29 such that the release feature 29 causes outer sheath 20 to tear along longitudinal axis “L” (or along any other suitable path) in a proximal direction, as indicated by arrow “X.” In this way, an opening (not explicitly shown) is formed in outer sheath 20, which enables removal of outer sheath 20 from occluder 40.

The second end portion 23 of outer sheath 20 may have a catch 23a configured to prevent the first end portion 31 of pull cord 30 from detaching from outer sheath 20 and/or release feature 29. Therefore, a continued application of the tensile force to pull cord 30 will pull outer sheath 20 from occluder 40 until outer sheath 20 is completely withdrawn from occluder 40 and the body orifice. As such, release feature 29 of outer sheath 20 and pull cord 30 may function together to facilitate removal of outer sheath 20 from occluder 40.

Continuing with reference to FIGS. 1 and 2, occluder 40 is configured to prevent the escape of insufflation gas or pneumoperitoneum from a body orifice. Occluder 40 includes an outer surface 41 that is in contact with inner surface 25 of outer sheath 20. Occluder 40 may be disposed between respective first and second end portions 21, 23 of outer sheath 20. As shown in FIGS. 1 and 2, occluder 40 is retained in a compressed state by outer sheath 20 and has a first dimension “D₁” when disposed within outer sheath 20 in this compressed state. In the compressed state, occluder 40 conforms to the shape of outer sheath 20 and is compressed by outer sheath 20 such that occluder 40 is loaded with potential energy.

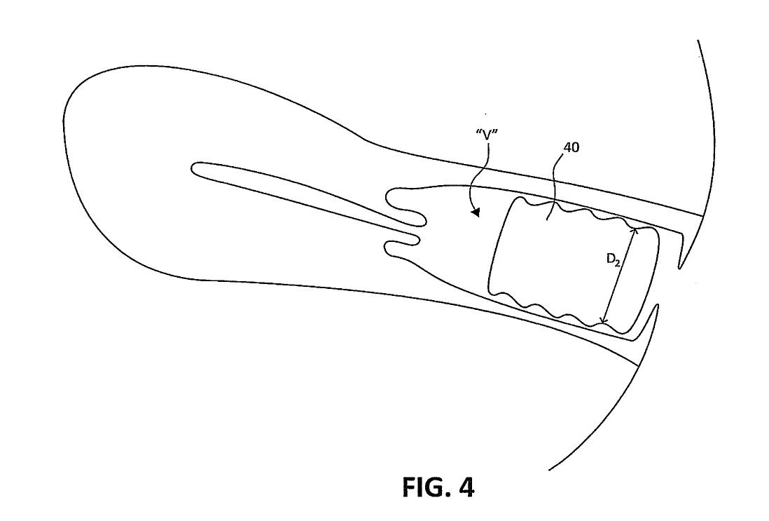

Once outer sheath 20 is removed from occluder 40, occluder 40 releases its potential energy and expands into an expanded state such that occluder 40 has a second, larger dimension “D₂” (FIG. 4). In the expanded state, occluder 40 is configured to conform to a body orifice (e.g., vaginal cavity) and form a fluid (e.g., air, gas, liquids, etc.) tight seal within the body orifice such that, for example, pneumoperitoneum cannot escape from the body orifice.

Occluder 40 may be formed from any suitable resiliently deformable material. For example, occluder 40 may be formed from a closed cell foam material, such as polyisoprene, polyethylene, cross-linked polyethylene, polypropylene, neoprene, polystyrene, etc. Occluder 40 may be a “memory” foam having sufficient compliance to form a seal about a body orifice (e.g., the vaginal cavity) upon insertion into the body orifice to conform therewith. A lubricious biocompatible coating, such as parylene N or C, may be applied to the outer surface 41 of occluder 40 in order to ensure that occluder 40 does not unnecessarily cause friction against a body orifice without diminishing the ability to establish a fluid-tight seal therewith. The lubricious coating on the outer surface 41 of the occluder 40 may also be configured to, e.g., facilitate removal of outer sheath 20 from occluder 40. Although occluder 40 is shown as having a cylindrical shape, occluder 40 may have any suitable shape, such as rectangular, square, triangular, elliptical, trapezoidal, or the like, to sufficiently seal a body orifice and to maintain pneumoperitoneum therein.

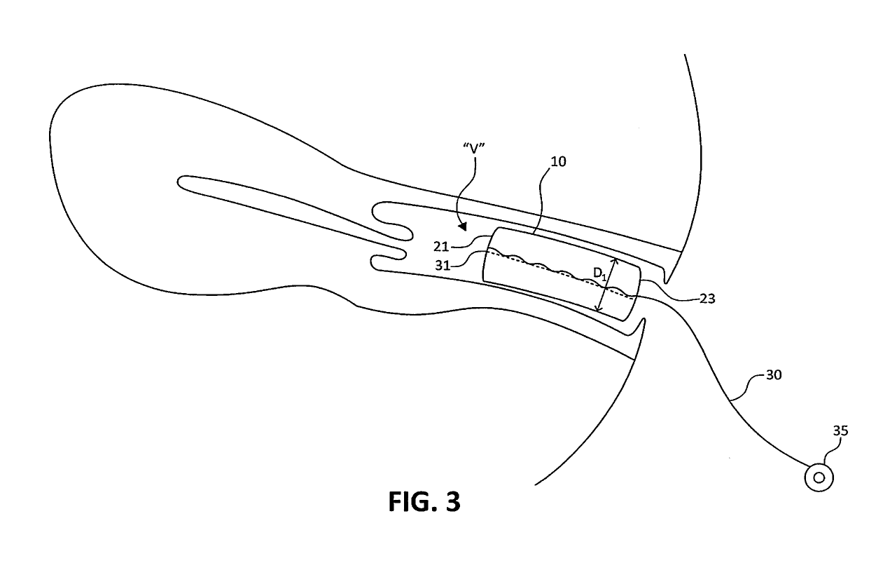

With reference to FIG. 3, in use, during for example, a laparoscopic hysterectomy, the abdominal cavity is insufflated with a gas, or pneumoperitoneum, to distend and separate the abdominal wall from the uterus, such that the clinician can adequately view the surgical site and perform, e.g., a colpotomy procedure. A distal end portion of expandable occlusion device 10 is inserted into a desired depth of a body orifice, for example, a vaginal cavity “V,” during and/or following a colpotomy in laparoscopic hysterectomy procedure. Initially, outer sheath 20 is disposed about and serves to compress occluder 40 into the first dimension “D₁” (FIGS. 1 and 2), such that expandable occlusion device 10 has a reduced profile for minimally invasive insertion into the vaginal cavity “V.” To deploy or expand the occluder 40 within the vaginal cavity “V,” a clinician takes hold of handle 35 of pull cord 30 and pulls pull cord 30 proximally or otherwise applies a tensile force in a proximal direction therewith. As the clinician applies this tensile force in the proximal direction, the first end portion 31 of pull cord 30 attached to outer sheath 20 or release feature 29 tugs at the outer sheath 20 such that an opening (not explicitly shown) is created in outer sheath 20, enabling outer sheath 20 and the pull cord 30 to be removed proximally from about occluder 40 and withdrawn from the vaginal cavity “V.”

With reference to FIG. 4, as outer sheath 20 is removed from occluder 40, occluder 40 expands to the second, larger dimension “D₂,” from the compressed state to the expanded state. In the expanded state, occluder 40 substantially conforms to and seals the vaginal cavity “V” such that insufflation gases used during the procedure to enlarge the area surrounding the surgical site do not escape through the vaginal cavity “V” and out of the patient. In this way, pneumoperitoneum is maintained and the clinician can continue or complete, e.g., the hysterectomy procedure. Upon completion of the procedure, occluder 40 may be removed (e.g., using surgical forceps, by hand, etc.).

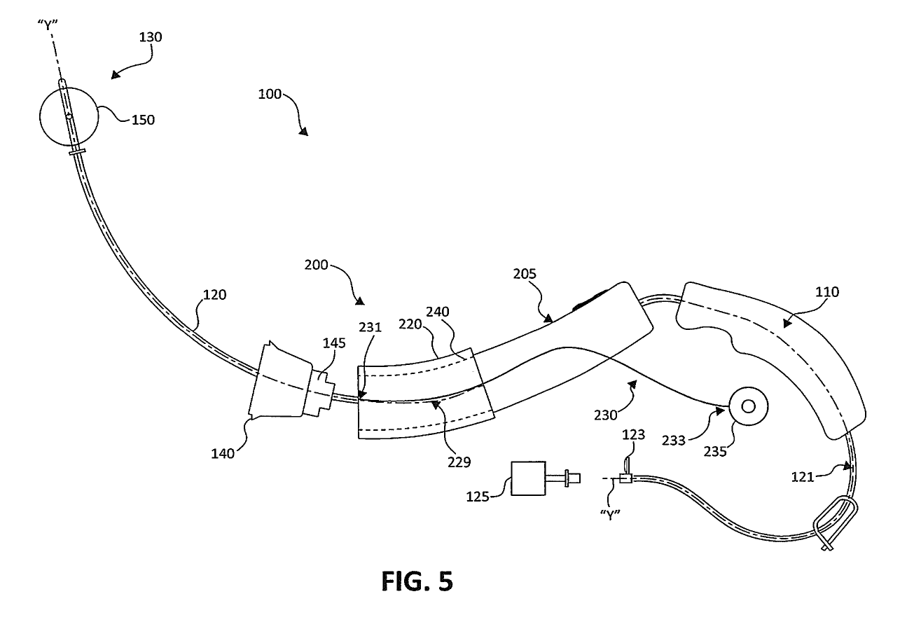

With reference to FIG. 5, in accordance with another embodiment of the present disclosure, a uterine manipulator is shown and generally identified by reference numeral 100. Uterine manipulator 100 is generally adapted for insertion through the vaginal cavity “V” and into the uterus “U” and is used to mobilize and/or position the uterus “U” during pelvic surgical procedures, e.g., laparoscopic hysterectomy. Uterine manipulator 100 defines a central axis “Y-Y” and generally includes a handle 110, a shaft 120 extending from handle 110, an expandable occlusion device 200 supported on a slidable occluder shaft 205, a uterine manipulating tip portion 130, a cervical cup 140, a shuttle 145, and an inflatable balloon 150.

Handle 110 of uterine manipulator 100 is configured for gripping and/or using uterine manipulator 100. Movement (e.g., pivoting, rotation, etc.) of handle 110 causes uterine manipulating tip portion 130 to move about central axis “Y-Y” for moving and/or positioning the uterus “U,” for example, for retroversion and anteversion of the uterus.

Shaft 120 extends distally from handle 110 and is generally an elongated tube configured for insertion through the vaginal cavity “V” and into the uterus “U.” Shaft 120 includes an actuation assembly (not shown) disposed therein, which is operatively connected to handle 110 at a proximal end portion thereof to enable manipulation of the uterus “U.”. Shaft 120 includes a fluid conduit 121 disposed therein that is connected to port 123 at a proximal end portion thereof and to inflatable balloon 150 at a distal end portion thereof. Port 123 couples to an inflation source 125 for delivering inflation fluid (e.g., saline, air, or the like) from inflation source 125 through fluid conduit 121 to inflatable balloon 150, such that inflatable balloon 150 may be inflated with the inflation fluid.

Expandable occlusion device 200 is similar to expandable occlusion device 10 described above, except that expandable occlusion device 200 is configured for use with uterine manipulator 100. Specifically, expandable occlusion device 200 includes a lumen (not explicitly shown) defined therethrough to receive slidable occluder shaft 205 of uterine manipulator 100 in sealing engagement thereabout. Slidable occluder shaft 205 may be movable proximally and/or distally along shaft 120.

Expandable occlusion device 200 includes an outer sheath 220, a pull cord 230 attached to outer sheath 220, and an occluder 240 disposed within outer sheath 220. Pull cord 230 is fixedly attached to outer sheath 220 and is configured to aid in removing outer sheath 220 from occluder 240. Pull cord 230 includes a first end portion 231 attached to outer sheath 220 and a second end portion 233 attached to a handle 235. A release feature 229 may be disposed along a length or surface of outer sheath 220 to facilitate removal of outer sheath 220 from occluder 240, similar to that as described above with respect to release feature 29 of outer sheath 20 (see FIG. 1).

Expandable occlusion device 200 may be formed separately from, or integrally with uterine manipulator 100. Expandable occlusion device 200 may be attached to any surface of uterine manipulator 100, such as any portion of slidable occluder shaft 205, shaft 120, or the like. Expandable occlusion device 200 may be rigidly attached to any surface of uterine manipulator 100 (e.g., via adhesives, fasteners, etc.). Alternatively, expandable occlusion device 200 may be slidably and/or frictionally engaged with any surface of uterine manipulator 100, such that expandable occlusion device 200 may be moved proximally or distally along uterine manipulator 100, or separated from uterine manipulator 100 as needed.

Although expandable occlusion device 200 is described herein for use with uterine manipulator 100, expandable occlusion device 200 may be adapted for use with any suitable surgical instrument. For example, the lumen (not shown) of expandable occlusion device 200 may be dimensioned to accommodate a shaft of any surgical instrument (not shown) for insertion into a body orifice. The surgical instrument may, for example, be an access instrument configured to receive additional instrumentation therethrough, may itself be configured to perform one or more surgical tasks, and/or may be configured to facilitate insertion and/or removal of expandable occlusion device 200.

Uterine manipulating tip portion 130 is configured for insertion into the uterus “U” and includes cervical cup 140 disposed on a proximal portion thereof and inflatable balloon 150 disposed at a distal end portion thereof. Cervical cup 140 generally defines a cup-like configuration configured to abut and envelope the cervix, e.g., for displacing the cervix away from the uterus, retracting the bladder, and/or defining cervical points of reference for a colpotomy incision. Shuttle 145 may be selectively coupled to cervical cup 140 to enable cervical cup 140 and shuttle 145 to move together. A distal end portion of uterine manipulating tip portion 130 includes inflatable balloon 150, which is configured to selectively inflate with inflation fluid from inflation source 125 for anchoring the uterine manipulator 100 within the uterus “U.”

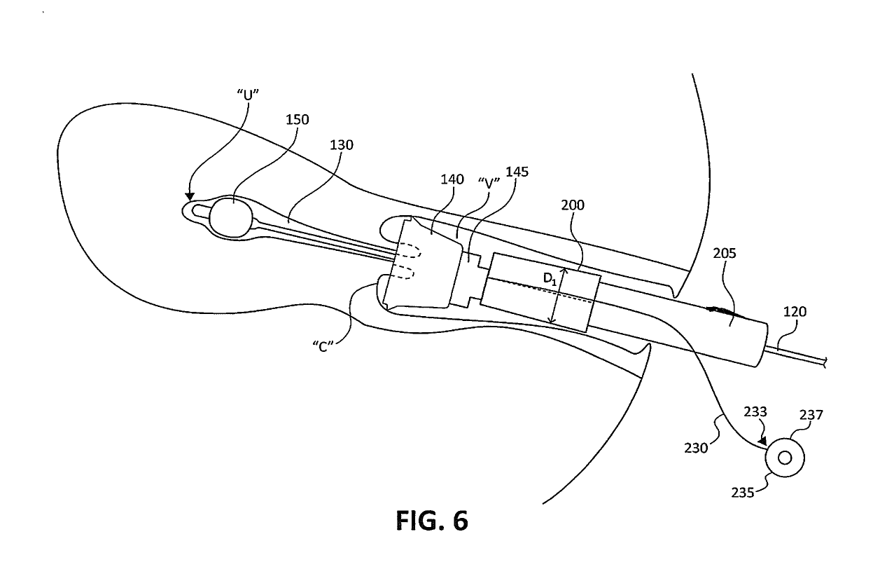

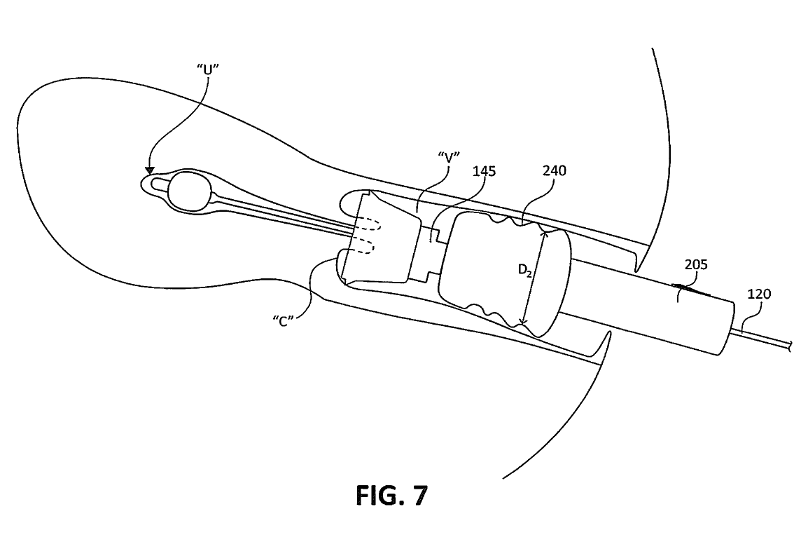

In use, during for example, a laparoscopic hysterectomy, the abdominal cavity is insufflated with a gas, or pneumoperitoneum, to distend and separate the abdominal wall from the uterus, such that the clinician can adequately view the surgical site and perform, e.g., a colpotomy procedure. With reference to FIG. 6, uterine manipulating tip portion 130 of uterine manipulator 100 is advanced distally through the vaginal cavity “V” of a patient until the cervical cup 140 is in abutment with the cervix “C” and the uterine manipulating tip portion 130 is placed within the uterus “U.”

Expandable occlusion device 200 is then disposed within the vaginal cavity “V” while in a compressed state having a first dimension “D₁′.” In the compressed state, expandable occlusion device 200 has a reduced profile for minimally invasive insertion into the vaginal cavity “V.” In this way, any friction from expandable occlusion device 200 within the vaginal cavity “V” does not affect or reduce the tactile feedback a clinician may use to position uterine manipulator 100 to a desired depth or location within the uterus “U.” The reduced profile of expandable occlusion device 200 in the compressed state prevents expandable occlusion device 200 from being pushed out by the body's natural pressures. In addition, expandable occlusion device 200 in the compressed state allows the clinician to visualize the surgical site (e.g., vaginal cavity “V,” cervix “C,” uterus “U,” etc.) without obstructing the surgical site to ensure that uterine manipulator 100 is inserted into a desired depth, position, and/or location.

Port 123 is coupled to inflation source 125 for delivering inflation fluid from port 123 through fluid conduit 121 to inflatable balloon 150 to inflate inflatable balloon 150. With inflatable balloon 150 of uterine manipulating tip portion 130 inflated, uterine manipulator 100 is anchored to the uterus “U” such that the uterus “U” can be positioned, e.g., for the colpotomy incision.

Movement of handle 110 causes movement of uterine manipulating tip portion 130, which causes movement of the uterus “U.” Specifically, movement of handle 110 causes movement of uterine manipulating tip portion 130 about central axis “Y-Y” for retroversion and anteversion of the uterus.

Once the uterus “U” is positioned as desired, the clinician may deploy the expandable occlusion device 200 by taking hold of handle 235 at the second free end portion 233 of pull cord 230 and applying a sufficient tensile force in a proximal direction therewith. As the clinician applies a tensile force to pull cord 230 in the proximal direction, the first end portion 231 of pull cord 230 attached to outer sheath 220 and/or release feature 229 tugs at the outer sheath 220 (and/or release feature 229 of outer sheath 220) until outer sheath 220 is removed from occluder 240.

As shown in FIG. 7, with outer sheath 220 removed from occluder 240, occluder 240 expands (e.g., by releasing latent potential energy) to the second, larger dimension “D₂” from the compressed state to an expanded state. In the expanded state, occluder 240 substantially conforms to, and seals the vaginal cavity “V” such that the insufflation gases used during the procedure to enlarge the area surrounding the hysterectomy target site, or pneumoperitoneum, do not escape through the vaginal cavity “V.” A clinician may then create a colpotomy incision (not shown) to, e.g., separate part or all of the uterus from the vagina. With the colpotomy created and the uterus separated from the vagina, the clinician may withdraw the uterine manipulator 100 from the vaginal cavity “V” with the severed uterus attached to the uterine manipulating tip portion 130.

With the uterine manipulator 110 removed from the vaginal cavity “V,” pneumoperitoneum must still be maintained in order for the clinician to maintain the surgical field of view during laparoscopy such that the clinician can seal off the vaginal cuff to complete the procedure. As can be appreciated, re-inserting a uterine manipulator into the vaginal cavity “V” in order to maintain pneumoperitoneum may increase the risk of inadvertent trauma to the patient's cervix “C,” intact sections of the uterus “U” (if any), or other tissue. Rather than re-inserting the uterine manipulator 100 into the vaginal cavity “V” to maintain pneumoperitoneum, the clinician may instead insert the expandable occlusion device 10 into the vaginal cavity “V,” as detailed above with respect to FIGS. 3 and 4. In this way, pneumoperitoneum is maintained quickly and effectively with minimal invasiveness to the patient. The clinician can then laparoscopically seal off the vaginal cuff to complete the hysterectomy. Once completed, the clinician can remove occluder 40 (FIGS. 3 and 4).

It should be appreciated that devices, systems, and methods described herein may be used for any type of surgical procedure and are not limited to laparoscopic hysterectomy procedures.

Persons skilled in the art will understand that the structures and methods specifically described herein and shown in the accompanying figures are non-limiting exemplary embodiments, and that the description, disclosure, and figures should be construed merely as exemplary of particular embodiments. It is to be understood, therefore, that the present disclosure is not limited to the precise embodiments described, and that various other changes and modifications may be effected by one skilled in the art without departing from the scope or spirit of the disclosure. Additionally, the elements and features shown or described in connection with certain embodiments may be combined with the elements and features of certain other embodiments without departing from the scope of the present disclosure, and that such modifications and variations are also included within the scope of the present disclosure. Accordingly, the subject matter of the present disclosure is not limited by what has been particularly shown and described.

What is claimed is:

1. An expandable occlusion device, comprising:
an expandable occluder expandable from a compressed state to an expanded state to sealingly engage tissue;
an outer sheath disposed about the expandable occluder and defining a longitudinal axis, the outer sheath configured to compress the expandable occluder and maintain the expandable occluder in the compressed state; and
a pull cord operably coupled to the outer sheath, wherein the pull cord is selectively manipulatable to tear the outer sheath such that the outer sheath is removed from the expandable occluder allowing the expandable occluder to expand from the compressed state to the expanded state.

2. The expandable occlusion device according to claim 1, wherein an application of tensile force to the pull cord tears the outer sheath and removes the outer sheath from the expandable occluder.

3. The expandable occlusion device according to claim 2, further comprising:
a release feature associated with a surface of the outer sheath and coupled to the pull cord, wherein when a tensile force is applied to the pull cord, a corresponding tensile force is applied to the release feature, the application of tensile force to the release feature tearing the outer sheath to form an opening in the outer sheath to facilitate removal of the outer sheath from the expandable occluder.

4. The expandable occlusion device according to claim 3, wherein the release feature is selected from the group consisting of tear tape, peel tape, and shrink tape.

5. The expandable occlusion device according to claim 3, wherein the release feature includes a series of perforations defined through a surface of the outer sheath.

6. The expandable occlusion device according to claim 1, wherein the outer sheath is formed from a biocompatible film.

7. The expandable occlusion device according to claim 1, wherein the expandable occluder is formed from a closed-cell memory foam.

8. The expandable occlusion device according to claim 1, wherein the pull cord includes first and second end portions, the first end portion fixedly attached to the outer sheath, the second end portion having a handle attached thereto, the handle configured for gripping the pull cord.

9. The expandable occlusion device according to claim 1, wherein one of the outer sheath and the expandable occluder includes a lubricious coating, the lubricious coating configured to aid at least one of insertion into a body cavity, removal from a body cavity, or removal of the outer sheath from the expandable occluder.

10. The expandable occlusion device according to claim 1, wherein the expandable occlusion device defines a circular cross-sectional shape.

11. The expandable occlusion device according to claim 1, wherein the expandable occlusion device is configured to receive a surgical instrument therethrough.

12. The expandable occlusion device according to claim 11, wherein the surgical instrument is a uterine manipulator.

13. A uterine manipulator, comprising:
a handle;
a shaft extending distally from the handle;
an end effector assembly disposed at a distal end portion of the shaft;
an expandable occlusion device supported on the shaft, including:
an expandable occluder expandable from a compressed state to an expanded state to sealingly engage tissue;
an outer sheath disposed about the expandable occluder and defining a longitudinal axis, the outer sheath configured to compress the expandable occluder and maintain the expandable occluder in the compressed state; and
a pull cord operably coupled to the outer sheath, wherein the pull cord is selectively manipulatable to tear the outer sheath such that the outer sheath is removed from the expandable occluder allowing the expandable occluder to expand from the compressed state to the expanded state.

14. The uterine manipulator according to claim 13, wherein the pull cord of the expandable occlusion device is of sufficient length to extend proximally outward from a vaginal cavity when the uterine manipulator is inserted into the vagina cavity.

15. The uterine manipulator according to claim 13, wherein when a tensile force is applied to the pull cord when the uterine manipulator is inserted into the vaginal cavity, the outer sheath is removed proximally outward from the vaginal cavity and the expandable occluder expands outwardly against the vaginal cavity to the expanded state to form a seal therein.

16. The uterine manipulator according to claim 13, further comprising:
a release feature associated with a surface of the outer sheath and coupled to the pull cord, wherein when a tensile force is applied to the pull cord, a corresponding tensile force is applied to the release feature, the application of tensile force to the release feature tearing the outer sheath to form an opening in the outer sheath to facilitate removal of the outer sheath from the expandable occluder.

17. A method for performing a surgical procedure, comprising:
positioning an expandable occlusion device within a surgical cavity in a compressed state, the expandable occlusion device including an expandable occluder, an outer sheath disposed about the expandable occluder to maintain the expandable occluder in the compressed state, and a pull cord operably coupled to the outer sheath;
applying a tensile force to the pull cord of the expandable occlusion device to remove the outer sheath from the expandable occluder such that the expandable occluder expands from the compressed state to an expanded state within the vaginal cavity to form a seal therein; and
insufflating a surgical site with a gas to create a pneumoperitoneum therein.

18. The method according to claim 17, further comprising:
positioning a uterus with a uterine manipulating tip portion of a uterine manipulator for at least one of retroversion and anteversion of the uterus.

19. The method according to claim 18, further comprising:
making a circular incision into the uterus to separate at least part of the uterus from a vagina; and
withdrawing the uterine manipulator proximally from the vaginal cavity with at least part of the uterus attached to the uterine manipulating tip portion of the uterine manipulator.

20. The method according to claim 19, further comprising:
positioning a second expandable occlusion device within a surgical cavity in a compressed state, the second expandable occlusion device including an expandable occluder, an outer sheath disposed about the second expandable occluder to maintain the second expandable occluder in the compressed state, and a pull cord operably coupled to the outer sheath;
applying a tensile force to the pull cord of the second expandable occlusion device to remove the outer sheath from the second expandable occluder such that the second expandable occluder expands from the compressed state to an expanded state within the vaginal cavity to form a seal therein; and
insufflating a surgical site with a gas to create a pneumoperitoneum therein.
